(19) United States
(12) Patent Application Publication
(10) Pub. No.: US 2014/0324021 A1
(43) Pub. Date: Oct. 30, 2014

(54) MEDICINAL PATCH AND INJECTOR SYSTEM

(71) Applicant: Eli Lilly and Company, Indianapolis, IN (US)

(72) Inventors: Michael Scott Ulrich, Columbus, OH (US); Andrew Vankirk Sweeney, Columbus, OH (US)

(73) Assignee: Eli Lilly and Company, Indianapolis, IN (US)

(21) Appl. No.: 14/355,586

(22) PCT Filed: Nov. 7, 2012
(86) PCT No.: PCT/US12/63874

§ 371 Date: May 1, 2014

Related U.S. Application Data
(60) Provisional application No. 61/556,542, filed on Nov. 7, 2011.

(51) Int. Cl. A61M 5/42

(52) U.S. Cl. CPC A61M 5/427; A61M 5/422; A61M 2205/3584; A61M 2205/33; A61M 2205/502; A61M 2205/6009; A61M 2205/6027
USPC 604/506; 604/116; 604/112

(57) ABSTRACT

An injection system including a wearable component (e.g., a patch) adapted to be worn by a user, and a medication delivery component (e.g., injection device) configured to cooperate with the wearable component to deliver medication to the user. The wearable component can provide a target area to the user for targeting the medication delivery component during use, and further serve to prepare the injection site to minimize the potential for injection site reactions. Both the wearable component and the medication delivery component can include electronics and/or other devices for detecting and/or communication information regarding usage of the injection system including dose amount, frequency, time, etc. The system can be configured to communicate such information to a third party, such as a health care professional.

### BACKGROUND

This application claims priority to U.S. Provisional Patent Application Ser. No. 61/556,542, filed on Nov. 7, 2011, the disclosure of which is hereby incorporated by reference in its entirety.

The present disclosure relates to medical devices. It finds particular application in conjunction with an injector system, and will be described with particular reference thereto.

Hypodermic syringes are often used to deliver selected doses of medication to patients. Such hypodermic syringes generally include a barrel and a plunger mounted for reciprocating movement within the barrel. A needle is mounted to the barrel and includes a cannula for receiving/delivering medication. Medication to be injected with the hypodermic syringe often is stored in a vial having a pierceable elastomeric seal. Medication in the vial is accessed by piercing the elastomeric seal with the needle. A selected dose of the medication is drawn into the barrel by withdrawing the plunger to create a vacuum in the barrel that is then filled by the medication. The needle is then withdrawn from the vial, inserted into a patient, and the medication is injected by moving the plunger in the opposite direction to expel the medicine from the barrel.

Some medications, such as insulin, are usually self-administered. Medication delivery pens have been developed to facilitate the self-administration of medication. Such delivery pens attempt to simplify both the delivery and dosing of the medication to make self-administering as reliable as administration by a healthcare professional. In addition, such devices often include various safety features to prevent or diminish the possibility of needle contamination and/or the needle making unintended contact with the user or another person.

To this end, various devices have been developed that simplify self-administration of medication. Reusable devices may accommodate a replaceable medication cartridge and include an injection mechanism for injecting a prescribed dose of medicine. Some devices are capable of varying the dosage size. Disposable pen devices generally have a fixed dosage of medication, and are designed for a single use. Many pen devices include safety mechanisms, such as a retracted needle that is shielded from inadvertent contact until deployed for injection.

### BRIEF DESCRIPTION

In accordance with one aspect of the present disclosure, an injection system includes a wearable component (e.g., a patch device) adapted to be worn by user, and a medication delivery component (e.g., an injection device) configured to cooperate with the wearable component to deliver medication to the user. The wearable component can provide a target area to the user for targeting the medication delivery component during use, and further serve to prepare the injection site to minimize the potential for injection site reactions. Both the wearable component and the medication delivery component can include electronics and/or other devices for detecting and/or communication information regarding usage of the injection system including dose amount, frequency, time, etc. The system can be configured to communicate such information to a third party, such as a health care professional.

In accordance with another aspect, a medication administration system comprises a wearable component adapted to be worn on a surface of a user's skin, and an injection component configured to cooperate with the wearable component to administer a medication to the user. The wearable component can include an adhesive patch for adhering the wearable component to the user's skin. The wearable component can be adapted to apply pain management to an injection site before an injection. The wearable component can include a housing having an opening for receiving the injection component.

The wearable component can further include a monitoring device for monitoring at least one parameter related to the use of at least one of the wearable component and the injection component. The monitoring device can include at least one sensor for sensing activity associated with use of at least one of the wearable component and the injection component, and/or a communication interface for communicating with the injection component. The communication interface can include an RFID reader, and the injection device can include an RFID transponder configured to be read by the RFID reader of the wearable component. The monitoring device can also include a communication interface for communicating data to a third party device, such as a mobile phone, a computer, an internet connected device or other remote device. A cover for covering at least a portion of the opening in the housing when the injection component is not received therein can be provided. The wearable component can include a user interface having at least one of a user input device, a display, or a sound producing device.

The injection component can include a housing, a portion of said housing adapted to be received in the opening in the housing of the wearable device. The injection device can also include an injection mechanism operative to inject medication into a user's body. At least one sensor associated with the injection device can be provided for detecting a parameter related to injection of medication via the injection mechanism.

In accordance with another aspect, a wearable medical device comprises a housing having a surface for engaging an outer surface of a user's skin, an opening in the housing for receiving an associated medication delivery device for injecting medication into a user's body, and a monitoring device for monitoring at least one parameter related to the use of at least one of the wearable component and the injection component.

These and other non-limiting characteristics are more particularly described below.

### BRIEF DESCRIPTION OF THE DRAWINGS

The following is a brief description of the drawings, which are presented for the purpose of illustrating the exemplary embodiments disclosed herein and not for the purpose of limiting the same.

FIG. 1 is a perspective view of an exemplary injection system in accordance with the disclosure.

FIG. 2 is a schematic block diagram of the exemplary injection system of FIG. 1.

FIG. 3 is a schematic block diagram of another exemplary injection system in accordance with the disclosure.

FIG. 4 is an illustration showing an injection device and a patch device in another exemplary embodiment of the present disclosure.

FIG. 5 is a perspective view of a patch device of the present disclosure prior to being applied to the user.

FIG. 6 is a top view of the patch device, illustrating the removal of the tab to expose the adhesive for applying the patch device to the user.

FIG. 7 is a side view of an injection device of the present disclosure.

FIG. 8 is a side view showing the injection device interacting with the patch device during medication administration.

### DETAILED DESCRIPTION

A more complete understanding of the processes and apparatuses disclosed herein can be obtained by reference to the accompanying drawings. These figures are merely schematic representations based on convenience and the ease of demonstrating the existing art and/or the present development, and are, therefore, not intended to indicate relative size and dimensions of the assemblies or components thereof.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function. In the following specification and the claims which follow, reference will be made to a number of terms which shall be defined to have the following meanings.

The singular forms “a,” “an,” and “the” include plural referents unless the context clearly dictates otherwise.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values). The endpoints of the ranges and any values disclosed herein are not limited to the precise range or value; they are sufficiently imprecise to include values approximating these ranges and/or values.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.”

The systems and methods of the present disclosure can be used with both manual syringes or auto-injectors and is not limited to cylindrical geometries. For the purposes of this disclosure, the term “injection device” is used to refer to both manual syringes and auto-injectors of any size or shape.

Certain disease treatment routines require frequent, sometimes daily, injections. Persons administering self-injection routines are often advised to rotate the injection site using four to six different sites. Frequent injections and side effects from medications can cause adverse reactions at injection sites. The present disclosure provides a system approach for self injection. The medication administration system is comprised of two main components; a device to administer injections, and a device to prepare the injection site to minimize the potential for injection site reactions.

As will be described in more detail below, the components of the system components contain electronics sensors, components, and/or wireless communication devices for the purpose of providing dose information and other usage data to software applications for reviewing dose history and making treatment management decisions.

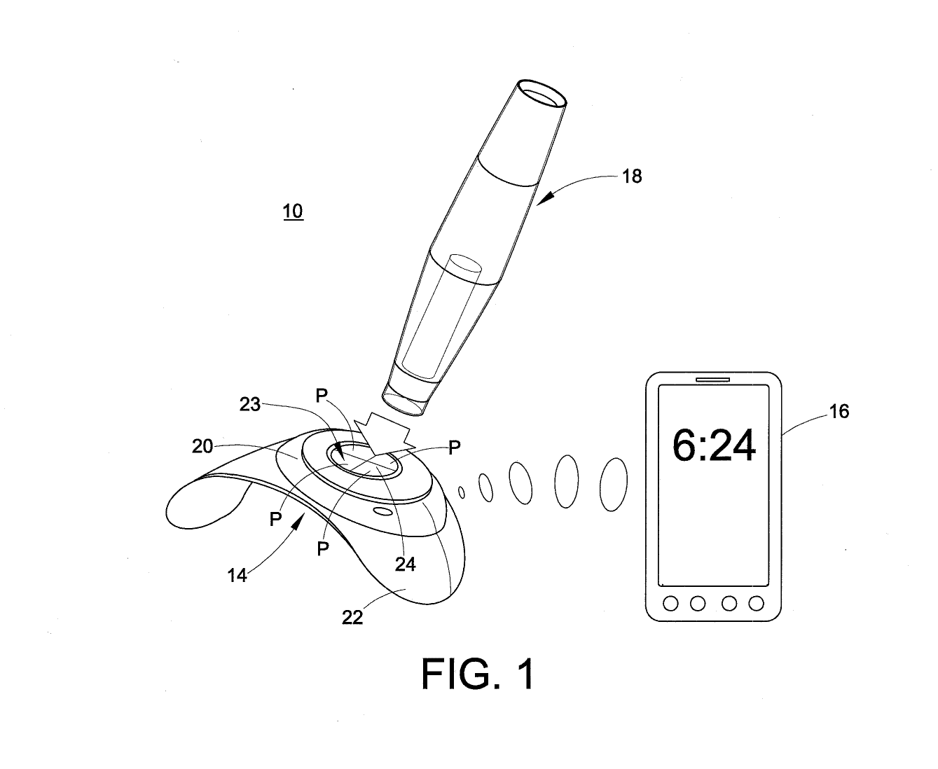

With reference to FIG. 1, an exemplary medication delivery system in accordance with the disclosure is illustrated and identified generally by reference numeral 10. The system 10 generally comprises a wearable component in the form a patch device 14, and an injection component in the form of an injection device 18 for dispensing medication. The patch device 14 is configured to transmit data associated with usage of the system to a third party device, such as smart phone 16, for example.

The patch device 14 is applied to the skin, using an adhesive, for example, at a predetermined injection site. The patch device 14 can contain medication to reduce pain from administering injections and/or to prepare the injection site to minimize side effects caused by frequent injections. The patch device 14 is a semi-durable device worn by the user for a short period of time, possibly up to two weeks, or more. The patch device 14 contains a target receptacle that provides an interface area for the user to inject medication through using an injection device, such as injection device 18. When the useful period of the patch device 14 expires, the device is removed from the skin and disposed. A new patch device may then be applied to the user for continuing the electronic management of medication.

The patch device 14 contains electronics and wireless communication devices and sensor mechanisms to provide additional treatment management features and capabilities to the end user. Sensors are incorporated into the patch device 14 for the purpose of communicating with the injection device 18, and for communicating with a software application that resides on a personal electronic device, such as a smartphone, iPad, computer or the like. The treatment information monitored and transmitted by the patch device can include the specific medicine contained in the injection device, the dose amount, time/date stamp of the injection, patient ID, etc.

The patch device 14 includes a housing 20, which is depicted here as being disc shaped. However, it should be noted that the housing may be of any desired shape. A flexible patch 22 is connected to the housing 20 and enables the wearable component to be secured to a user's body, such as an arm, a leg, a portion of the torso or any other suitable location for injecting medication. The patch 22 can be coated with a suitable adhesive for adhering the patch device 14 to a surface of the user's skin. As will be appreciated, the flexibility of the patch 22 allows the patch device 14 to be worn on a wide variety of body parts and also facilitates user movement of body parts on which the patch device 14 is secured. For example, a patch device 14 secured to a user's forearm or bicep may be subjected to stress due to expansion and contraction of the user's muscles during everyday activities. The flexible patch 22 accommodates such expansion and contraction while maintaining adhesion to the surface of the user's skin.

The housing 20 of the wearable component (patch device 14) as noted above can be disc-shaped and includes an opening 23 that is configured to receive the injection device 18 during a medication injection procedure. The opening is usually centrally located. The opening 23 can be closed by a cover 24 that, as illustrated here, is designed to part upon insertion of the injection device 18 into the opening 23. As such, the injection site can be shielded from exposure to the environment by the housing 20 and cover 24 before and after an injection procedure.

The cover 24 is comprised of four portions P (e.g., flaps) that meet together at a central location within the opening 23. Each of these portions P are flexible and secured about the circumference of the opening 23 such that, upon insertion of the injection device 18 into the opening 23, the portions P deflect downwardly to create an aperture at the center of the opening 23 through which the injection device 18 can deliver the medication. Upon withdrawal of the injection device 18 from the opening 23 in the patch device 14, the portions P resiliently return to their original position thereby closing off the opening 23 and shielding the injection site from the environment.

As noted, the wearable component 14 can also be configured to deliver and/or apply pain management to the injection site prior to an injection procedure. For example, the patch 22 of the patch device 14 can be saturated with (or otherwise be designed to deliver) a topical anesthetic for numbing the injection site. As will be appreciated, it may be advantageous to apply the topical anesthetic to only a portion of the patch 22, such as the portion adjacent the opening 23. The patch device can also be configured to deliver or apply anti-inflammatory or other drug types for managing the injection site so as to prolong the viability of a given injection site.

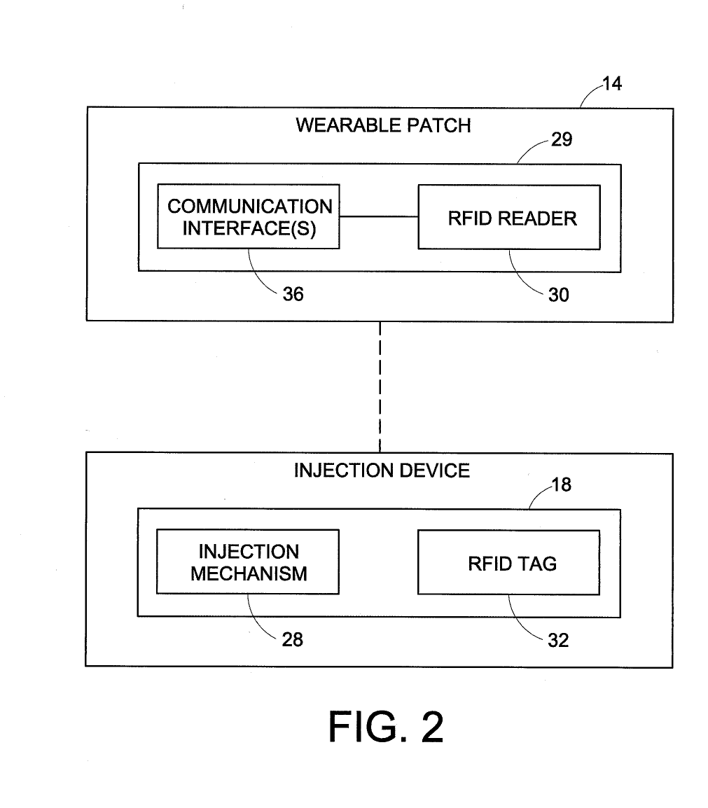

Turning now to the details of the injection device 18, it will be appreciated that the exemplary device is generally a pen-shaped structure. The injection device 18 has an operative end that is configured to be received in the opening 23 of the patch device 14 (as indicated by the arrow) to deliver medication to the user. To this end, the injection device 18 includes an injection mechanism 28 (see FIG. 2) that is operative to inject medication into a user's body, such medication being stored in an medicine reservoir or the like.

In some embodiments, the injection device can be a single-use injection device containing a reservoir for liquid medication, a needle and an injection mechanism, and user interface for activation. Onboard sensors and electronic components may be embedded in the injection device for wireless communication with the patch device. Some of the types of information that may be transmitted include device ID number and dose information including amount and time stamp, for example. As another example, for injection devices that can vary the dosage size, the patch device can be used to confirm that the correct dosage is being applied to reduce overdosing.

Although the illustrated injection device 18 is designed to work in conjunction with the patch device 14, it will be appreciated that other injection components not necessarily designed to operate with the wearable component of the present disclosure can be used with the patch device 14. However, in such cases, some of the features that will be described below may not be available to a user using an existing rejection device. The details of the injection component insofar as they relate to the injection of medication into a user's body through the user's skin are not necessarily germane to the present disclosure and therefore the details of such mechanisms have been omitted for the sake of brevity. It will be appreciated, however, that a wide variety of injection mechanisms can be used without departing from the scope of the present disclosure.

As noted above, the patch device 14 further includes a monitoring device 29 (or devices) for monitoring at least one parameter related to the use of the patch device 14 and/or the injection device 18. The monitoring device may include one or more electrical components and or mechanical switches designed to sense or detect certain events or activity associated with use of the system 10.

For example, referring now to FIG. 2, in one basic configuration the patch device 14 can include an RFID reader 30 for interrogating an RFID tag 32 associated with the injection device 18. A communication interface 36, which may be a wireless communication interface (WiFi, Bluetooth, etc.) is provided for establishing a communication link between the patch device 14 and another device or devices, such as a smart phone as shown in FIG. 1, and/or cell phones, laptops and other like devices. The communication interface 36 can be used for communicating data relating to the usage of the system 10 to another device or system

The RFID reader 30 can be configured to interrogate the RFID tag 32 during an injection procedure. For example, a switch or other mechanical or electrical device can be provided on the patch device 14 for detecting when the injection device 18 is inserted into the opening 23. When such an event is detected, the patch device 14 can be configured to interrogate the RFID tag 32 and to receive information therefrom such as medicine type, dosage amount etc. This information, along with other information such as time/date etc. can be communicated to a third party device via the communication interface 40.

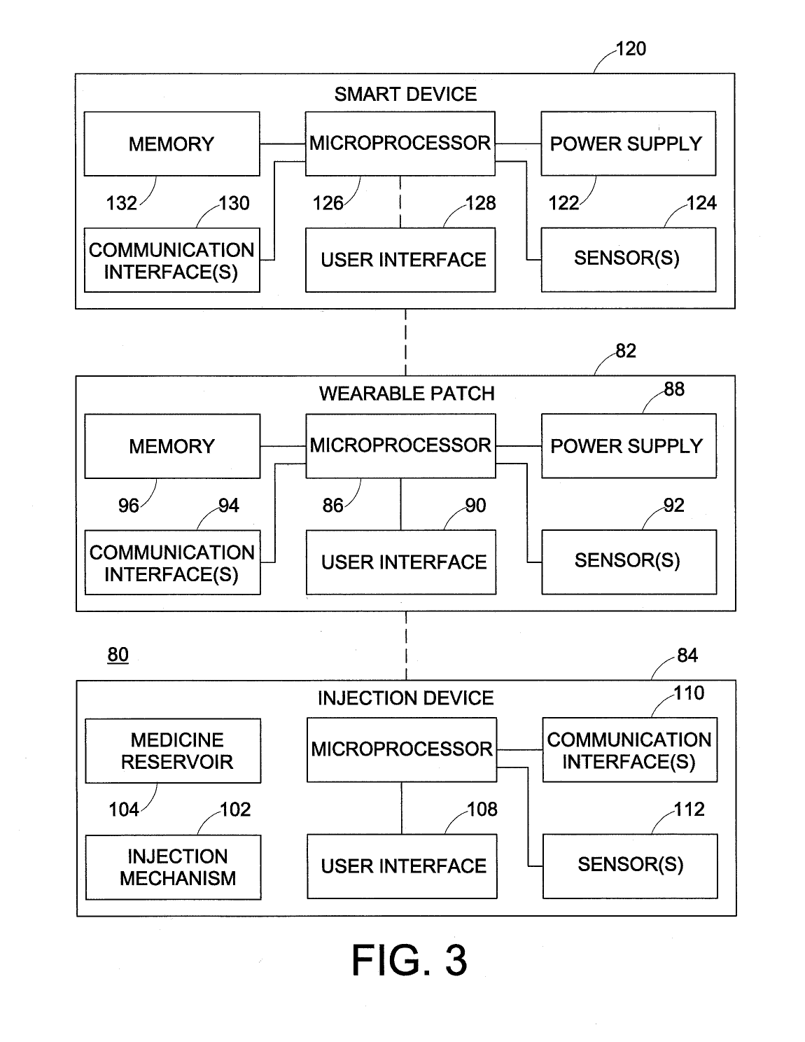

FIG. 3 illustrates a somewhat more sophisticated system 80 including a wearable component 82 and an injection component 84. Here, the wearable component includes a plurality of electrical components designed to work together to sense one or more parameters related to the use of the system and/or report or otherwise communicate data relating to the sensed parameters to a third party smart device 120 such as a personal computer, a smartphone, a tablet, a laptop, or any other similar device.

As illustrated here, the wearable component 82 includes a microprocessor 86 connected to a power supply 88, which may be an onboard battery, AC power supply or the like. The wearable component 82 also includes a user interface 90, one or more sensors 92 for detecting activity relating to usage of the system 80, and a communication interface 94 for communicating with the injection device 84 and/or a third party or remote device such as those listed above. Onboard memory 96 is also provided for storing data related to the usage of the system 80 and/or software for performing the various functions, etc.

Similarly, the injection component 84 in the embodiment illustrated in FIG. 3 includes a medicine reservoir 102 operatively connected to an injection mechanism 104 for delivering medication to the user. The injection component 84 can further include various electronics for enhancing the functions of the component. To this end, the illustrated injection component 84 includes a microprocessor 106 operatively connected to a user interface 108, a communication interface 110, and one or more sensors 112. It will be appreciated, however, that the injection component 84 need not include any of the electrical components shown in FIG. 2. For example, the injection device can utilize a simple RFID transponder that is designed to be read by a corresponding RFID reader located in the wearable component 82, such as in the system of FIG. 2.

The wearable component 82 can be configured to communicate with the injection component 84 to share information related to the operation of the system 80. For example, the injection component 84 can communicate information such as the device ID, the patient ID, the medication, the dose volume, the recommended frequency for administering the medication, etc. Such information can be communicated to the wearable component 82 upon the first use of the injection component 84 with the wearable component 82. A unique injection component identifier may also be provided to the wearable component 82 in order to allow the wearable component 82 to differentiate between different injection components 84.

The wearable component 82 can be configured to sense or otherwise determine when an injection procedure has been performed and record data related to the injection procedure in the memory. For example, upon insertion of an injection component 84 into the wearable component 82, the wearable component 82 may record the time and date of the sensed injection procedure. Combined with information received from the injection component, such as medicine type, dosage amounts, etc., a timeline can be constructed that illustrates a user's self administration of medication. The data used to construct such timeline may be transmitted to a third party via a variety of means. The wearable component may also provide scheduled prescription injection reminders to the user, for example through sight (e.g. blinking lights) or sound (e.g. audible alarm).

For example, and returning to FIG. 1, the wearable components described herein can communicate with a smartphone 16 via Bluetooth or other suitable connection to relay data from the wearable component to the smartphone. The smartphone 16 can then forward such information across the Internet or directly to a healthcare provider for review by a healthcare professional. By utilizing existing communication devices such as smart phones, personal computers and the like, the present disclosure facilitates real-time communication of information relating to patient self-administration of medicine to be monitored by health professionals thus improving patient medical history records and possibly patient compliance with treatment regimens.

Returning to FIG. 3, the wearable component 82, as noted above, also includes a user interface 90. The user interface 90 can include at least one of a user input device such as a button or buttons, a display and/or an audible alarm. The user interface 90 can be used for configuring the wearable component 82. For example, a certain patient may need to administer a medication four times a day and the wearable component can be configured to remind a user approximately six hours after the last dose of medication was administered, to administer the next dose of medication. In the event that more than six hours lapses since the last administration of medication, the wearable component, having not sensed an injection procedure, may sound an alarm or otherwise indicate to the user the need to administer the next dose of medication. After an extended time lapses past the next scheduled dose, and if no injection procedure has been detected by the wearable component, the wearable component may send a signal to the smartphone which may be forwarded to a healthcare professional as noted informing the healthcare professional that the patient has missed their dose of medicine. The healthcare professional can then call or otherwise contact the user to determine the cause for the delay in administration of the scheduled dose.

As noted above, the injection component may simply contain an RFID tag. Such RFID tag may include a unique identifier and/or other information that allows the wearable component to determine certain information about the injection component. For example, the injection component could include an RFID tag having information regarding the type of medication and the dosage size of the medication. In such case, the injection component may be a single use disposable injection component where a user uses the component a single time injecting the entire payload of medication and then discards the injection component. In one embodiment, the wearable component can be configured to scan the RFID tag when an injection procedure is detected. To this end, a switch or other detection device may be provided within the opening of the housing of the wearable component. Such switch may be activated upon insertion of an injection component whereby an associated RFID tag reader in the wearable component would scan the RFID tag in the injection component and receive the information contained thereon.

The third party smart device 120 also includes a microprocessor 126 connected to a power supply 122, which may be an onboard battery, AC power supply or the like. The smart device 120 also includes a user interface 128, one or more sensors 124, and a communication interface 130 for communicating with the wearable component 82. Onboard memory 132 is also provided for storing data related to the usage of the system 80 and/or software for performing the various functions, etc.

As will now be appreciated, the present disclosure sets forth a wearable patch that can, among other things, apply pain management and track injection history, be used for multiple injections, provide reminders for scheduled injections (e.g., programmable), provide a target area for injection, and communicate information relating to usage of the device (e.g., injection history etc.) to health care personnel.

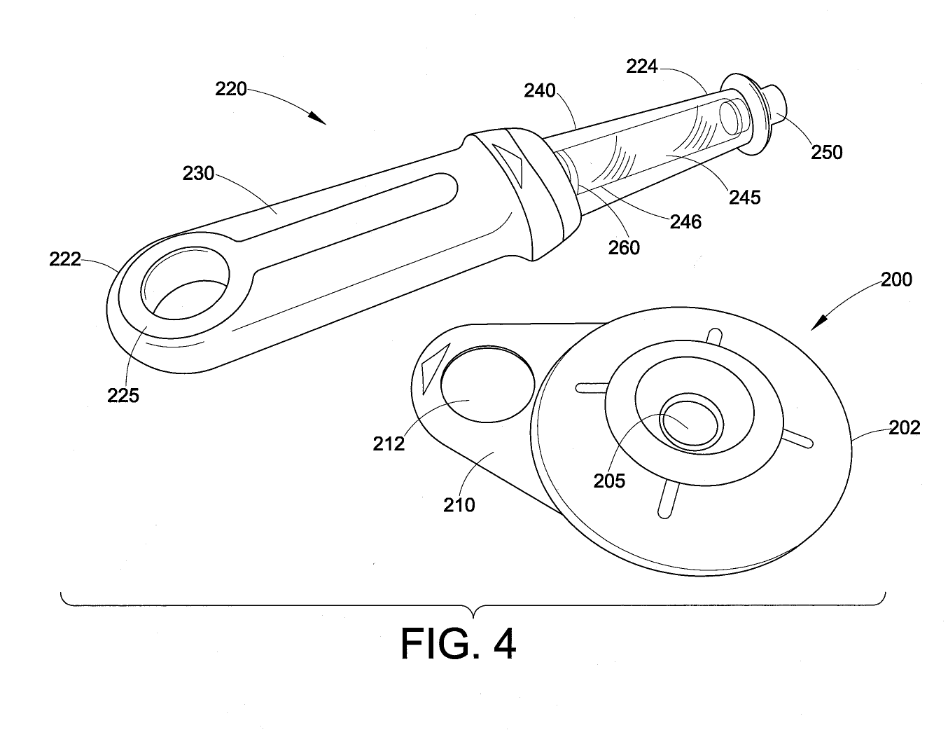

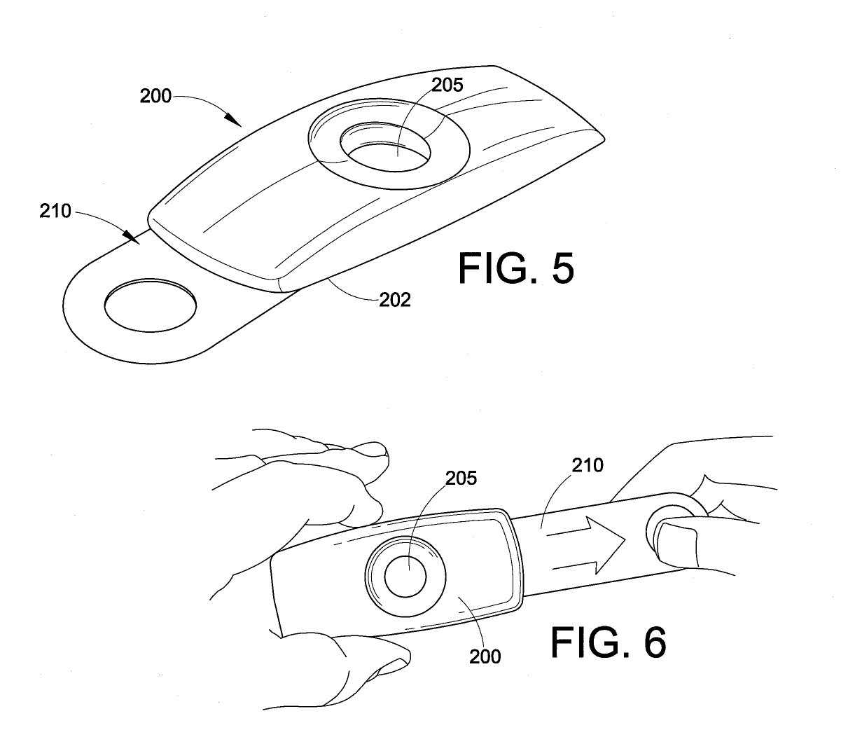

FIG. 4 is a perspective view of another exemplary medication administration system of the present disclosure. The injection device (i.e. syringe) and the patch device (i.e. wearable component) are visible here. Additional aspects of the system and the devices are visible in FIGS. 5-8.

Turning first to the patch device 200, the patch device again has a circular or disc-shaped perimeter 202. An opening 205 is located in the patch device. The opening is contoured to be complementary to the needle end 224 of the injection device. A pull tab 210 is visible extending from one side of the perimeter of the patch device. The pull tab includes a large hole 212 through which the user's fingers can be inserted to grasp the pull tab and expose the adhesive on the bottom of the patch device for attachment to the skin. The pull tab itself can be made from a hypoallergenic plastic film or other known materials.

Referring now to FIG. 5, a different embodiment of the patch device 200 is illustrated. Initially, this embodiment of the patch device has a rectangular perimeter 202. The opening or injector interface 205 is again contoured to be complementary to the needle end of the injection device. The opening is in a central location. It is contemplated in some embodiments that the injector interface may contain an embedded magnet (not visible), which can be used to engage and secure the injection device during use. FIG. 6 is a top view showing the pull tab 210 being used to expose adhesive on the bottom of the patch device. The central opening 205 is also visible.

In embodiments, the patch device may have a diameter of from about 2 inches to about 3 inches. As may be recognized, this size permits, if desired, a number of such patch devices to be placed on the user's body. This can be useful in permitting the user to rotate the injection site being used, further reducing adverse reactions.

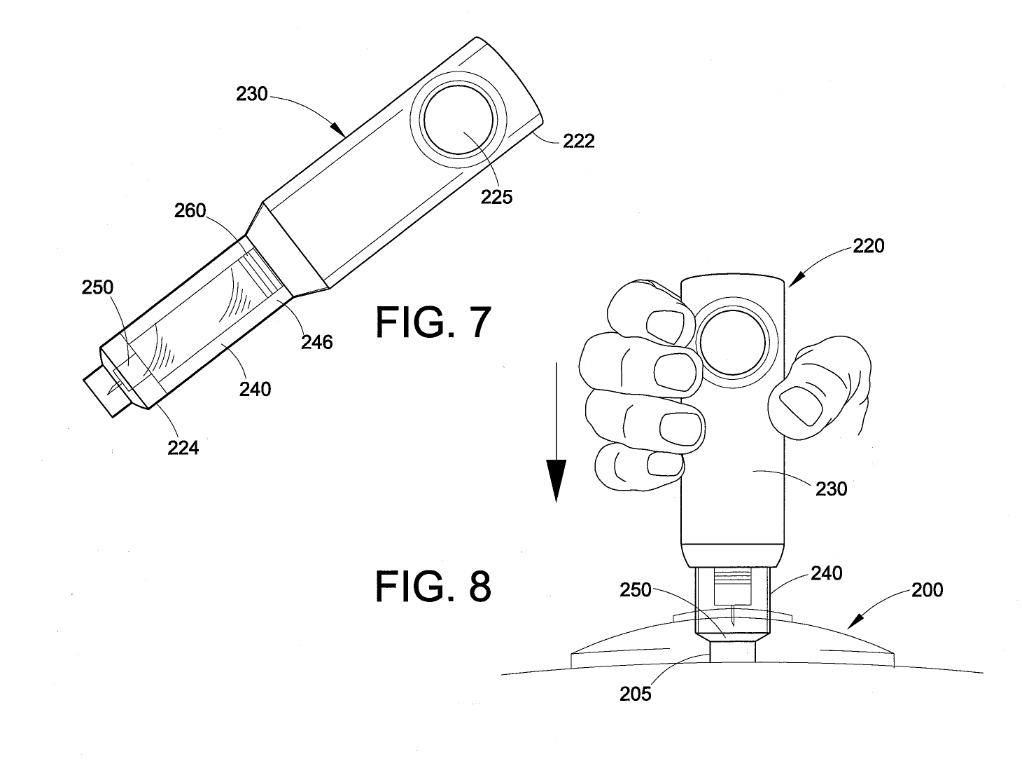

Referring now to FIG. 4 and FIG. 7, the injection device 220 generally includes a handle 230, a barrel 240, and a base 250. The injection device may be described as having a handle end 222 and a needle end 224. A portion of the barrel is visible here, with the remainder of the barrel being located within the handle. The barrel is illustrated here as being transparent so that some inner components are visible.

The handle 230 is shaped to be useful to users with deficiencies and limitations in manual dexterity, coordination, and strength, for example users with symptoms of rheumatoid arthritis (RA), multiple sclerosis (MS), and other such conditions/diseases. For example, here the handle is sized to be easily grasped, and includes a ring-shaped grip 225. The base 250 of the injection device contains the needle (not visible) through which medication is dispensed. The base is also shaped to be complementary to the opening of the patch device. The barrel 240 includes the various components needed to dispense medication. For example, in FIG. 7, a fluid chamber 245 is visible inside the barrel in which the medication is stored. The plunger 260 is located at the upper end 246 of the fluid chamber.

FIG. 8 is a schematic illustration showing the injection device 220 and patch device 200 being used to dispense medication, with the patch device being shown in a cutaway. As seen here, the base 250 of the injection device interfaces with the opening 205 next to the user's skin. The handle 230 is then depressed downwards relative to the barrel 240 in order to activate the mechanism that depresses the plunger and delivers the medication. The needle (not visible) automatically inserts and retracts.

Materials and methods for making the various components of the systems disclosed herein are known in the art.

The processes and devices described herein may be used to deliver a high-viscosity fluid containing protein microparticles made using the processes described in U.S. Provisional Patent Application Ser. No. 61/556,047, filed Nov. 4, 2011, the disclosure of which is hereby incorporated by reference in its entirety. The devices described herein may also use the core annular flow processes and devices described in U.S. Provisional Patent Application Ser. No. 61/556,491, filed on Nov. 7, 2011, and in U.S. Provisional Patent Application Ser. No. 61/673,864, filed on Jul. 20, 2012, the disclosures of which are hereby incorporated by reference in their entirety. The devices described in U.S. Provisional Patent Application Ser. No. 61/556,709, filed Nov. 4, 2011, the disclosure of which is hereby incorporated by reference in their entirety, can also be used in the systems of the present disclosure.

The present disclosure has been described with reference to exemplary embodiments. Obviously, modifications and alterations will occur to others upon reading and understanding the preceding detailed description. It is intended that the present disclosure be construed as including all such modifications and alterations insofar as they come within the scope of the appended claims or the equivalents thereof.

1. A medication administration system comprising:
a wearable component adapted to be worn on a surface of a user's skin;
an injection component configured to cooperate with the wearable component to administer a medication to the user.

2. The system of claim 1, wherein the wearable component includes an adhesive patch for adhering the wearable component to the user's skin.

3. The system of claim 2, wherein the patch is adapted to apply pain management to an injection site before an injection.

4. The system of claim 1, wherein the wearable component includes a housing having an opening for receiving the injection component.

5. The system of claim 4, further comprising a cover for covering at least a portion of the opening in the housing of the wearable component when the injection component is not received therein.

6. The system of claim 4, wherein the injection component includes a base, the base being adapted to be received in the opening in the housing of the wearable component.

7. The system of claim 1, further comprising a monitoring device for monitoring at least one parameter related to the use of at least one of the wearable component and the injection component.

8. The system of claim 7, wherein the monitoring device includes at least one sensor for sensing activity associated with use of at least one of the wearable component and the injection component.

9. The system of claim 7, wherein the monitoring device includes a communication interface for communicating with the injection component.

10. The system of claim 9, wherein the communication interface includes an RFID reader.

11. The system of claim 10, wherein the injection component includes an RFID transponder configured to be read by the RFID reader.

12. The system of claim 7, wherein the monitoring device includes a communication interface for communicating data to a third party device.

13. The system of claim 12, wherein the third party device includes at least one of a mobile phone, a computer, and an internet connected device.

14. The system of claim 12, further comprising the third party device.

15. The system of claim 1, wherein the wearable component includes a user interface having at least one of a user input device, a display, or a sound producing device.

16. The system of claim 1, wherein the injection device includes an injection mechanism operative to inject medication into a user's body.

17. The system of claim 1, further comprising at least one sensor for detecting a parameter related to injection of medication via the injection mechanism.

18. A wearable medical device comprising:
a housing having a surface for engaging a user's skin;
an opening in the housing for receiving an associated medication delivery device for injecting medication into the user's body; and
a monitoring device for monitoring at least one parameter related to the use of at least one of the wearable component and the associated injection component.

19. A method of injecting medication comprising:
applying a wearable component to a surface of an injection site;
injecting medication using an injection component configured to be received by the wearable component; and
communicating data related to operation of at least one of the wearable component and the injection component.
